(19) United States
(12) Patent Application Publication
(10) Pub. No.: US 2025/0009338 A1
(43) Pub. Date: Jan. 9, 2025

(54) SYSTEMS, DEVICES, AND ASSOCIATED METHODS FOR THERAPEUTIC EUS ANCHOR

(71) Applicant: Thomas Jefferson University, Philadelphia, PA (US)

(72) Inventor: Alexander Schlachterman, Merion, PA (US)

(21) Appl. No.: 18/706,936

(22) PCT Filed: Nov. 2, 2022
(86) PCT No.: PCT/US22/79132

§ 371 Date: May 2, 2024

Related U.S. Application Data
(60) Provisional application No. 63/353,207, filed on Jun. 17, 2022.
(60) Provisional application No. 63/275,788, filed on Nov. 4, 2021.

(51) Int. Cl. A61B 8/00; A61B 17/00; A61B 17/04; A61M 25/00; A61M 25/04

(52) U.S. Cl. CPC A61B 8/445; A61B 17/0467; A61B 17/0469; A61B 2017/00818; A61B 2017/0409; A61B 2017/0417; A61B 2017/0464; A61M 2025/0004; A61M 2025/0095; A61M 25/0097; A61M 25/04

(57) ABSTRACT

An endoscopic ultrasound (EUS) anchoring system is provided. The EUS anchoring system includes a needle sheath defining a sheath lumen. The EUS anchoring system also includes an endoscopic cannula translatable within the sheath lumen and defining a cannula lumen. The EUS anchoring system also includes an anchor translatable within the cannula lumen and egressable from a distal end of the endoscopic cannula. The EUS anchoring system also includes a wire suture translatable through the cannula lumen having a distal end coupled to a middle portion of the anchor. The EUS anchoring system also includes a cinch mechanism translatable within the sheath lumen and configured to: (a) cut the wire suture; and (b) couple a proximal end of the a cut wire suture still coupled to the anchor with a cinch.

### CROSS-REFERENCE TO RELATED APPLICATIONS

The present application claims priority under 35 U.S.C. § 119 (e) to U.S. Provisional Patent Application No. 63/353,207, filed Jun. 17, 2022, and U.S. Provisional Patent Application No. 63/275,788, filed Nov. 4, 2021, the content of both of which are incorporated herein by reference in their entirety.

### BACKGROUND OF THE INVENTION

Endoscopic ultrasound (EUS) is a minimally invasive procedure to assess digestive and lung conditions. An endoscope incorporating an ultrasound emitter can be inserted into a patient's esophagus, where the ultrasound emitter can emit ultrasonic waves that can be sensed to create detailed images of the patient's organs, digestive tract, and the like.

### SUMMARY

One aspect of the disclosure provides an endoscopic ultrasound (EUS) anchoring system. The EUS anchoring system includes a needle sheath defining a sheath lumen. The EUS anchoring system also includes an endoscopic cannula translatable within the sheath lumen and defining a cannula lumen. The EUS anchoring system also includes an anchor translatable within the cannula lumen and egressable from a distal end of the endoscopic cannula. The EUS anchoring system also includes a wire suture translatable through the cannula lumen having a distal end coupled to a middle portion of the anchor. The EUS anchoring system also includes a cinch mechanism translatable within the sheath lumen and configured to (a) cut the wire suture and (b) couple a proximal end of a cut wire suture still coupled to the anchor with a cinch.

This aspect can have a variety of embodiments. The EUS anchoring system can further include a plunger coupled to a proximal end of the endoscopic cannula and configured to apply a compressive force on the wire suture. The endoscopic cannula can include a 19 gauge needle, a 22 gauge needle, or a combination thereof. The suture wire can include 3.0 gauge polypropylene or 2.0 gauge polypropylene.

The EUS anchoring system can include an ultrasonic probe coupled to a distal end of an EUS scope. The EUS anchoring system can include a hooked wire translatable within a clamp lumen defined by a clamp and a distal end of the hooked wire comprising a hook, wherein the hooked wire is configured to: couple to the wire suture via the hook; and position the wire suture into the clamp lumen by translating the hook distally away from the anchor. The EUS anchoring system can include an EUS scope defining a EUS lumen, wherein the needle sheath is translatable within the EUS lumen.

The needle sheath can further define a fluid injection port divergent from a distal portion of the needle sheath. The fluid injection port and the distal portion can form a Y connect.

The endoscopic cannula can further define a plurality of apertures along a distal portion of the endoscopic cannula.

The EUS anchoring system can include: a second anchor translatable within the cannula lumen and egressable from the distal end of the endoscopic cannula; and a second wire suture translatable through the cannula lumen having a distal end coupled to a middle portion of the second anchor. The anchor and the second anchor can be predisposed at different positions along a length of the cannula lumen.

A length of the anchor can be parallel to a length of the cannula lumen when disposed within the cannula lumen. A length of the anchor can be perpendicular to a length of the cannula lumen when the clamp cuts the wire suture. The cinch mechanism can be housed within a handle of the endoscopic cannula.

Another aspect of the disclosure provides a system including: an anchor; a suture coupled to a medial portion of the anchor; an outer cannula defining an outer-cannula central bore; and an inner cannula defining an inner-cannula central bore. The inner-cannula central bore has a cross-sectional dimension configured to accommodate the suture. The outer-cannula central bore has a cross-sectional dimension configured to accommodate the inner cannula. The inner cannula and the outer cannula each have a length greater than about 1 m. The inner cannula further comprises a proximal stop shoulder at a proximal end, the proximal stop shoulder having a cross-sectional profile larger than the outer cannula to prevent the proximal end from entering the outer cannula.

This aspect can have a variety of embodiments. The system can further include a removable stopper adapted and configured to be removably mounted on an outer surface of the inner cannula to prevent advancement of the inner cannula and the anchor distal to the inner cannula from advancing within the outer cannula.

Another aspect of the disclosure provides a method of using the EUS anchoring system as described herein. The method includes: (a) directing an EUS scope towards a first section of tissue, thereby positioning a needle sheath, an endoscopic cannula, a wire suture, and an anchor, within the EUS scope, such that the respective distal ends are in a desired position away from the section of tissue; (b) positioning the anchor to be external to the EUS scope; (c) passing the anchor and the endoscopic cannula into tissue layers; (d) inserting the needle sheath, the wire suture, and the anchor into a second section of tissue, the anchor exiting the endoscopic cannula; and (e) removing the endoscopic cannula from the tissue layers, thereby leaving the anchor and the wire suture in the second section of tissue.

This aspect can have a variety of embodiments. The method can further include (f) flowing a fluid through the needle sheath and into the second section of tissue. The method can further include: (g) removing the needle sheath from the second section of tissue; (h) feeding a cinch over the wire suture along with a pusher tube; (i) pulling the anchor the tissue layers by pulling the proximal end of the wire suture; and (j) removing the needle sheath and the pusher tube.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a gastrointestinal system having a lumen apposing metal stent (LAMS).

FIG. 2 depicts a therapeutic EUS anchoring device according to an embodiment of the present disclosure.

FIG. 3 depicts cinches that are couplable to an anchor and by a therapeutic EUS anchoring device according to an embodiment of the present disclosure.

FIG. 4 depicts an anchor of a therapeutic EUS anchoring device according to an embodiment of the present disclosure.

FIG. 5 depicts a handle and needle sheath for a therapeutic EUS anchoring device according to an embodiment of the present disclosure.

FIGS. 6 and 7 depict anchors coupled to a suture for a therapeutic EUS anchoring device according to an embodiment of the present disclosure. The anchors include a recessed proximal end that receives the suture when within a sheath. After advancement distally out of the sheath, proximal tension on the suture causes the anchor to rotate perpendicular to the suture and engage with tissue on opposite sides of the opening through which the anchor passed.

FIG. 8 depicts a handle, needle sheath, and anchor for a therapeutic EUS anchoring device according to an embodiment of the present disclosure.

FIG. 9 depicts portions of a therapeutic EUS anchoring device according to an embodiment of the present disclosure across several steps. In the first view from the left, the deployable anchor is still engaged with the handle and can be advanced through tissue to be anchored. In the second view from the left, the anchor has been advanced and rotated perpendicular to the suture. The anchor may be held under tension against the tissue to be anchored (not depicted). A cinching device is proximal to the anchor and a sheath. In the third view from the left, the cinch is advanced distally with a pusher tube. In the right-most view, the cinch has passed through the outer sheath and can engage with an opposing tissue surface, e.g., by pressure from the pusher tube.

FIGS. 10A-10J depicts a process for implanting an anchor into a patient via a therapeutic EUS anchoring device according to an embodiment of the present disclosure.

FIG. 11 depicts a cross-sectional perspective view of a cinch implemented by a therapeutic EUS anchoring device according to an embodiment of the present disclosure.

FIGS. 12A-12D provide cross-sectional side views of a system according to an embodiment of the invention.

FIGS. 13A and 13B provide top views of exemplary removable stoppers according to embodiments of the invention.

FIGS. 14 and 15 provide photographs of proximal and distal ends of system 1100 according to embodiments of the invention. The outer cannula is held by a proximal handle in FIG. 14. The inner cannula 1110 is removed from the outer cannula 1106 in FIG. 15, but the relative size difference can be seen.

FIG. 16 illustrates a deployable EUS anchor s-lock, in accordance with certain exemplary embodiments of the present disclosure.

FIGS. 17A-17C illustrate certain exemplary embodiments of the present disclosure as used in the body. FIG. 17A illustrates GL LAMS with anchors and cinches. FIG. 17B illustrates a Roux-en-Y/EDGE with anchors and cinches. FIG. 17C illustrates a linear EUS used in connection with a 2.0 M long suture deployed through a gastric lining and jejunum.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

### Therapeutic EUS Anchoring Device

The disclosure described herein relates to a therapeutic EUS anchoring device and associated methods.

Various medical procedures involving a patient's gastrointestinal tract incorporate the use of LAMS for the creation of fistulae. For example, EUS-directed transgastric ERCP (EDGE), Roux-en-Y gastric bypass, gastro-jejunostomy, and the like, can implant or install a LAMS to create and maintain a fistula size and location. However, in some cases, the LAMS can migrate from the original placement in the gastrointestinal tract, which can ultimately lead to internal perforation, infection, sepsis, bleeding, emergent surgery, and/or death.

The therapeutic EUS anchoring devices described herein can implant or install an anchor with the aid of EUS. The anchor can be placed alongside a LAMS within a patient's gastrointestinal tract to secure the opposing GI walls to one another. The anchor can mitigate or prevent the GI walls from migrating from one another, which can decrease the chances of LAMS dislodgment and associated consequences from dislodgment (e.g., pain, internal bleeding, and the like). In some cases, multiple anchors can be implanted or installed in different locations between the opposing walls, which can further secure the GI walls to each other. In some cases, the implanting or installing of the anchors can allow for the removal of the LAMS after a period of time (e.g., a month, and the like). A fistula can be left opened or closed based on the purpose of the LAMS placement.

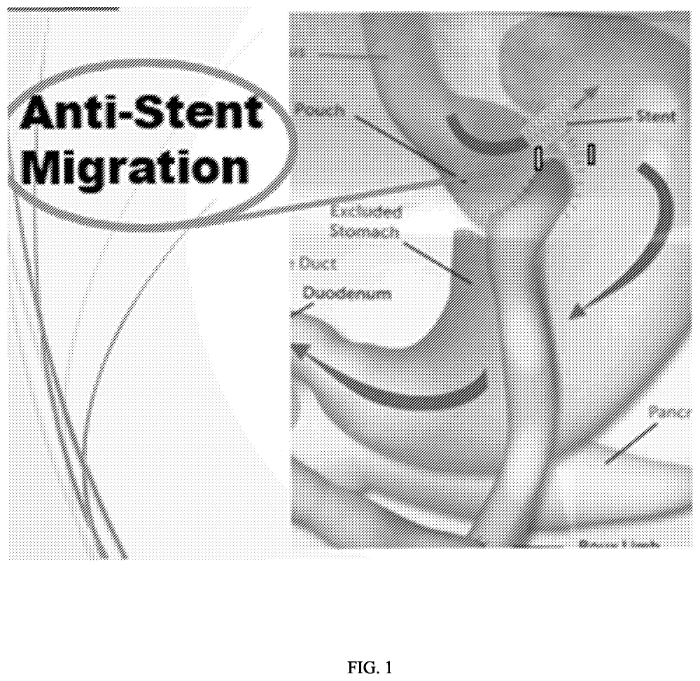

Referring now to the drawings, FIG. 1 depicts a GI system of a patient that underwent a gastric bypass procedure. As shown, the patient's stomach and duodenum is bypassed, such that the esophagus is coupled to the jejunum via a stomach pouch. The channel formed by the stomach pouch and the excluded stomach is created by a fistula, which can itself be created through an implanted LAMS. However, as discussed above, the LAMS can migrate in some cases, which can cause adverse side effects or subsequent remedial surgery. Installation or implantation of anchors between the opposing stomach pouch wall and excluded stomach wall, and via a therapeutic anchoring device, can mitigate the chances of LAMS migration.

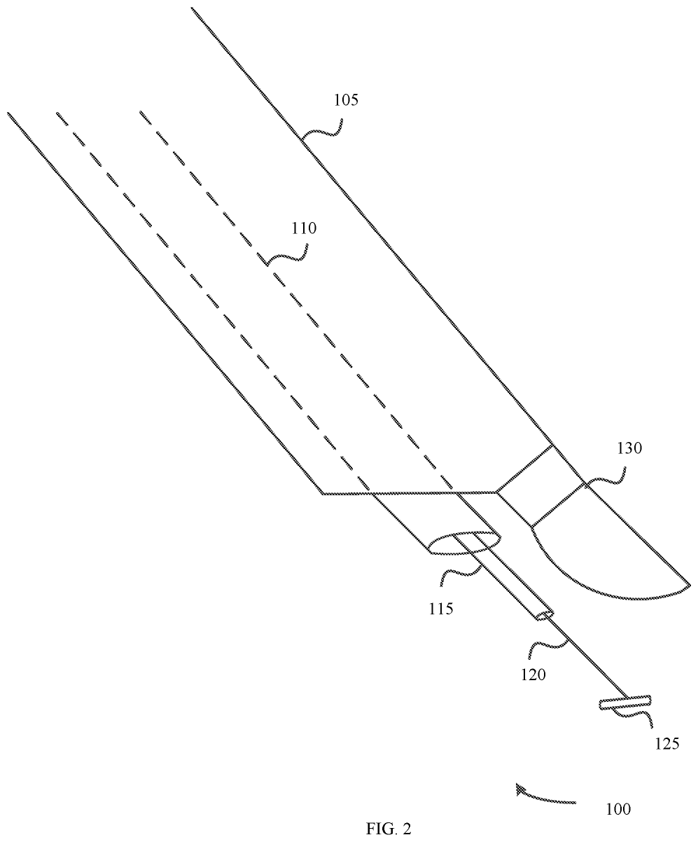

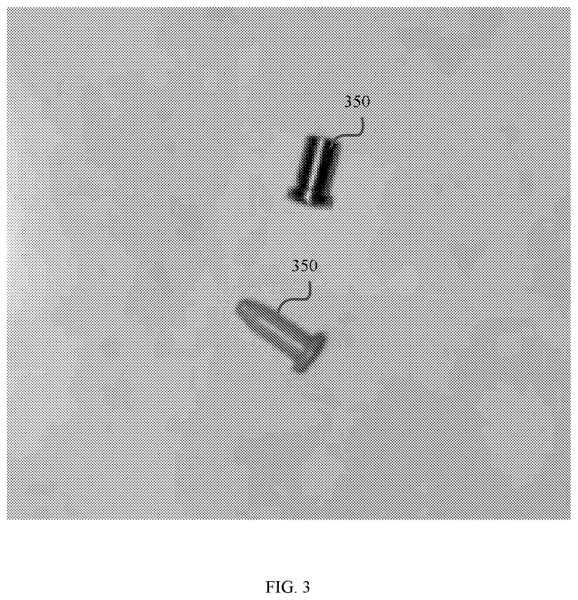

FIG. 2 depicts a therapeutic EUS anchoring device 100 according to an embodiment of the present disclosure. The anchoring device 100 can include an EUS scope 105, a needle sheath 110, an endoscopic cannula 115, a wire suture 120, an anchor 125, and an ultrasonic probe 130.

### EUS Scope

The EUS scope 105 can include a length and cross-sectional diameter sufficient to enter and traverse a patient's GI tract. For example, the length of the EUS scope 105 can vary based on the intended position of the distal end of the EUS scope 105 in a patient's GI tract. For example, a shorter EUS scope 105 may be used for a GI tract that underwent a gastric bypass surgery, as opposed to an EUS scope 105 having a longer length to reach a possible gallstone or pancreatic condition (e.g., during EDGE).

The EUS scope 105 can define a lumen that spans the length of the EUS scope 105. The length of the EUS scope (upper GI scope length). Roux en Y gastric by pass anatomy changes lumen access due to longer length to reach the major papilla for ERCP access of the biliary system. EDGE procedure with LAMS from the Jejunum or gastric pouch to the remnant (excluded) stomach, allows access to the major papilla.

The distal end of the EUS scope 105 can include an ultrasonic probe 130. The ultrasonic probe 130 can emit ultrasonic wavelengths that can be received by an ultrasonic sensor (e.g., which may be a part of the ultrasonic probe 130). In some cases, the ultrasonic probe 130 can be a radial probe, which can emit wavelengths in a radial direction. Diagnostic EUS Radial scope allows for non-invasive evaluation of surrounding organs, lymph nodes, vessels or structures adjacent to the lumen of the GI tract. Therapeutic Linear EUS scope allows for non-invasive evaluation of surrounding organs, lymph nodes, vessels or structures adjacent to the lumen of the GI tract as well but also has a channel for passing devices. Devices such as needles, anchor-cinches, and the like, can be passed through this channel.

### Endoscopic Cannula

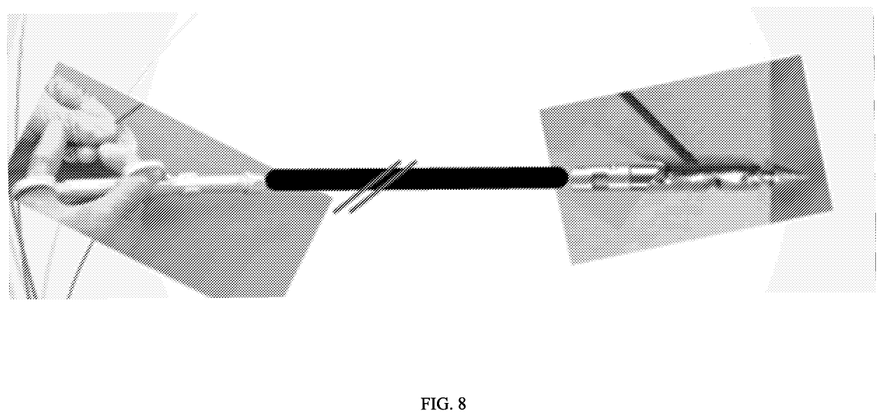

The therapeutic EUS anchoring device can also include an endoscopic cannula 115. The endoscopic cannula 115 can include such size and shape sufficient to translate through the needle sheath 110. For example, the endoscopic cannula can include a length greater than that of the EUS scope 105, so that an operator can control the translation and positioning of the distal end of the endoscopic cannula 115 via the proximal end of the endoscopic cannula 115 and independent of the positioning of the EUS scope 105. For example, the endoscopic cannula 115 can be between a 26 gage and a 16 gage cannula. Further, the distal end of the endoscopic cannula can be variously shaped. For example, in some cases the distal end can be flat or blunted (e.g., for use with a tipped anchor). In other cases, the distal end may be pointed (e.g., to puncture through the GI walls for implanting the anchor 125). The endoscopic cannula can also define a cannula lumen through the length of the endoscopic cannula 115. For example, in some cases, the endoscopic cannula 115 can be an endoscopic needle. FIG. 8 depicts an endoscopic cannula 115, with an anchor 125 disposed at the cannula distal end, and a handle for operator control at the proximal end.

### Anchor

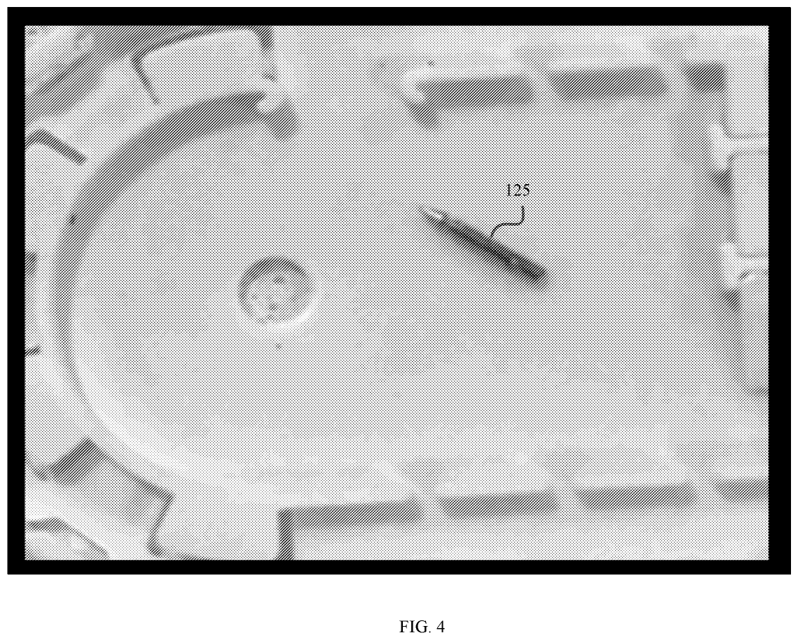

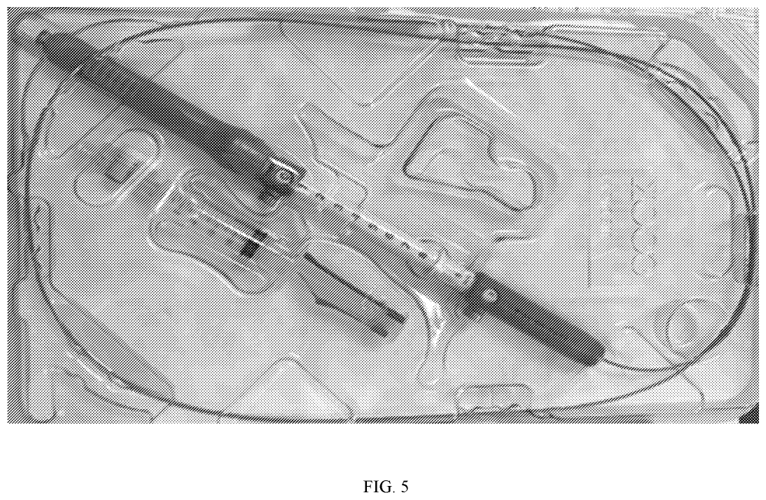

The therapeutic EUS anchor device can also include an anchor 125. The anchor 125 can include a cross-sectional diameter sufficient to translate through the cannula lumen while the anchor length is positioned parallel to the length of the cannula lumen. The anchor 125 can include a first end, a second end, and a middle portion. In some cases, the first end, the second end, or both, can be rounded, blunted, or flat. In some cases, the first end or the second end can be pointed (e.g., which can puncture the GI walls during implantation), such as the anchor 125 depicted in FIG. 4.

### Wire Suture

The therapeutic EUS anchor device can also include a wire suture 120. The wire suture 120 can include a diameter sufficient to translate through the cannula lumen. The wire suture 120 can also include a length sufficient for the distal and proximal ends of the wire suture to be external to the cannula lumen, while the remaining length to be disposed within the cannula lumen (e.g., for an operator to translate the wire suture independently from the positioning of the cannula). For example, the wire suture 120 can be composed of 3.0 Polypropylene, 2.0 (3.0 metric) Polypropylene, stainless steel, silk, nylon, polyester, and the like, and can have a length that extends past the length of the linear EUS endoscope length (e.g., greater than or equal to 230 cm).

The distal end of the wire suture 120 can be coupled to the middle portion of the anchor 125. For example, the coupling can occur through tying the wire suture 120 to the anchor 125, fusing the distal end of the wire suture 120 to the anchor 125, and the like. This coupling can allow for the anchor 125 to reposition itself with respect to the length of the wire suture 120, and may be dependent on whether the anchor 125 is internal to or external to the cannula lumen. For example, the anchor 125 may be disposed within the cannula lumen such that the length of the anchor 125 is parallel to the length of the cannula lumen. When the anchor 125 exits the cannula lumen such that the entire body of the anchor 125 is external to the cannula 115, the anchor 125 may hinge around the coupling with the wire suture 120. Thus, once the anchor 125 exits the cannula 115, the anchor positioning may transition from being parallel to the length of the cannula 115, to being perpendicular to the length of the cannula 115.

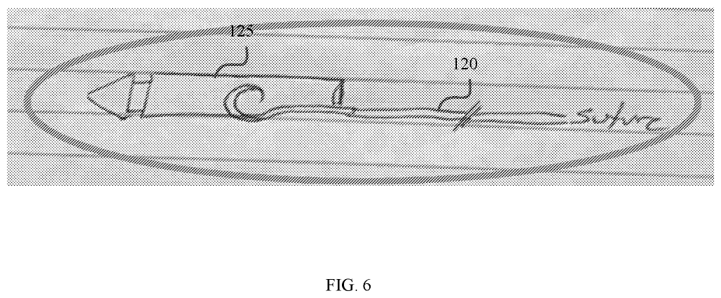

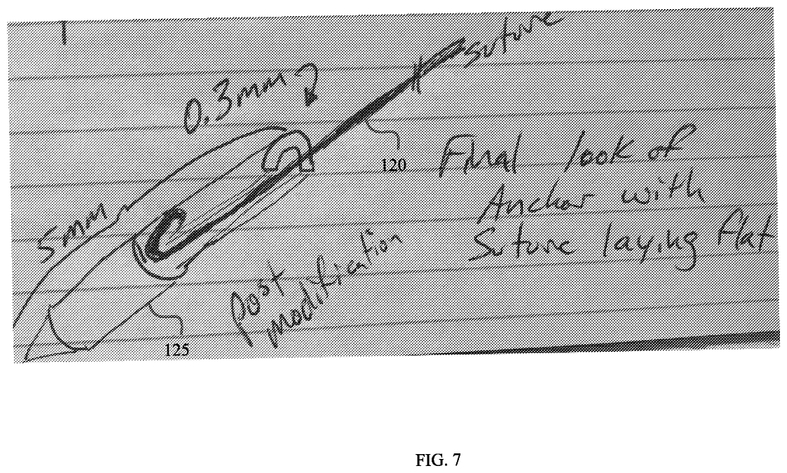

In some cases, the shape of the anchor 125 can facilitate this anchor reorientation. For example, one of the ends of the anchor 125 may include a proximal cross-sectional thickness that is smaller than the opposing distal end, such as in the anchors 125 depicted in FIGS. 6 and 7. In some cases, the proximal anchor end may form a channel for the distal end of the wire suture 120 to be disposed in when the anchor 125 is in the cannula lumen.

### Cinch

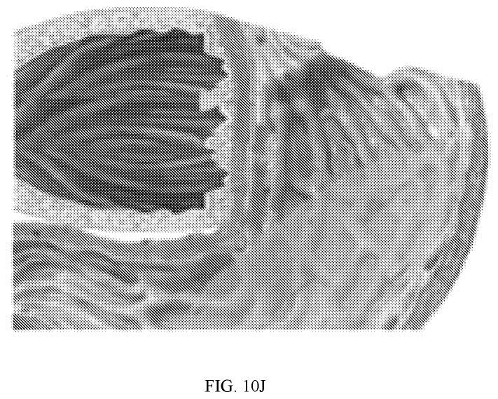

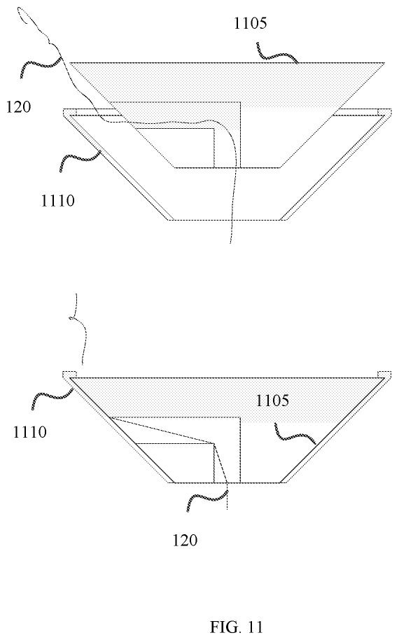

The therapeutic EUS anchor device can also include a cinch 350. The cinch 350 may couple to the anchor 125 via the wire suture 120. The cinch 350 can define a lumen (e.g., for the wire suture 120 to translate through). The cinch 350 can include a first end and a second end. In some cases, the first end may include a lip or rim that has a larger cross-sectional diameter than the cinch 350 (e.g., for preventing the cinch 350 to pass through the GI tract walls that the anchor 125 passed through). Coupling of a cinch to cut and “fix” the end of the suture preventing suture slipping, holds suture/anchor in place. The cinch pass down the Linear EUS channel and/or be encompassed in the anchor delivery system passing down the endoscopic cannula 115. There are various lengths, diameters, and shapes (cylinder, hexagon, rectangular for the cinch) that the cinch can be composed of, and on skilled in the art would understand the particular shapes and dimensions the cinch may take according to its intended use. An example of a cinch that can be implemented by the therapeutic EUS anchor device is depicted in FIG. 11. In this example, the cinch includes a first portion 1105 and second portion 1110. The wire suture (e.g., wire suture 120) can be threaded through an aperture defined by the second portion 1110 and also threaded through one or more apertures defined by the first portion 1105 such that the wire suture 120 is disposed between an exterior surface of the first portion 1105 and an interior surface of the second portion 1110. The first portion 1105 and the second portion 1110 can be compressed together, which can cut the wire suture 120 and cinch the wire suture 120 between the portions of the cinch.

### Cinch Mechanism

The therapeutic EUS anchor device can also include a cinch mechanism, such as cinch mechanism. The cinch mechanism can include a length and cross-sectional diameter sufficient to translate through the EUS scope lumen (e.g., such that the positioning of the cinch mechanism can be independent of the positioning of the EUS scope 105).

The cinch mechanism can define a cinch lumen. The cinch lumen can span the length of the cinch mechanism, and can be of size sufficient for a wire suture 120 to translate through. The cinch mechanism can also include a distal end. The distal end can include a mechanism to cut the wire suture 120. The proximal end of the cinch mechanism can include a plunger or other mechanism to initiate the cutting mechanism of the distal end.

In some cases, the cinch mechanism can include a handle and housing that houses multiple aspects of the anchor device. The cinching mechanism can include a body defining a lumen terminating at distal and proximal ends. The cinching mechanism can also include an anchor pusher handle disposed along the body of the cinching mechanism. The body of the cinching mechanism can be translatable through the anchor pusher handle, such that when the body translates in a distal fashion (e.g., the anchor pusher handle travels towards the proximal end of the body), the body of the cinch mechanism can push an anchor housed in the body out of the distal end of the cinching mechanism.

Further, the distal end of the cinching mechanism can include an egress for the anchor and wire suture, and an injection port for injecting fluid into a patient cavity. The egress can provide for an outlet for the anchor and coupled wire suture to exit the cinching mechanism. Further, in some cases, the cinching mechanism body can facilitate the translation of a cinch over the wire suture and to the distal end of the wire suture. For example, in some cases, the body can be translated distally away to eject an anchor from the body. The body can then be retracted, and a cinch can be disposed within the body (e.g., over the coupled wire suture). The body can then be translated through the pusher handle to translate the cinch towards the ejected anchor. Further compressive force can cause the cinch to cut and cinch the wire suture.

### Process

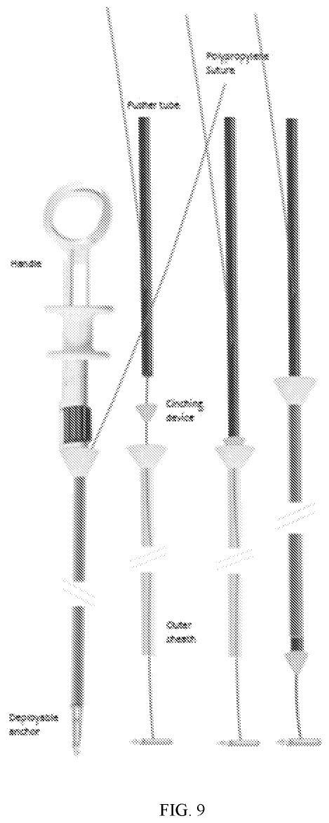

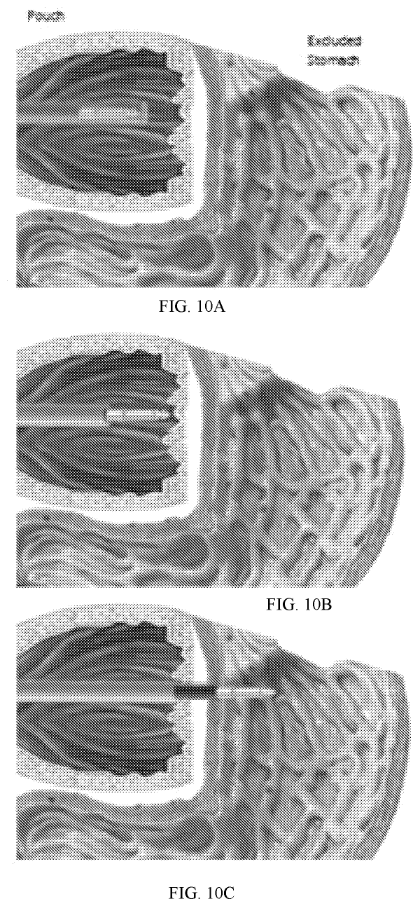

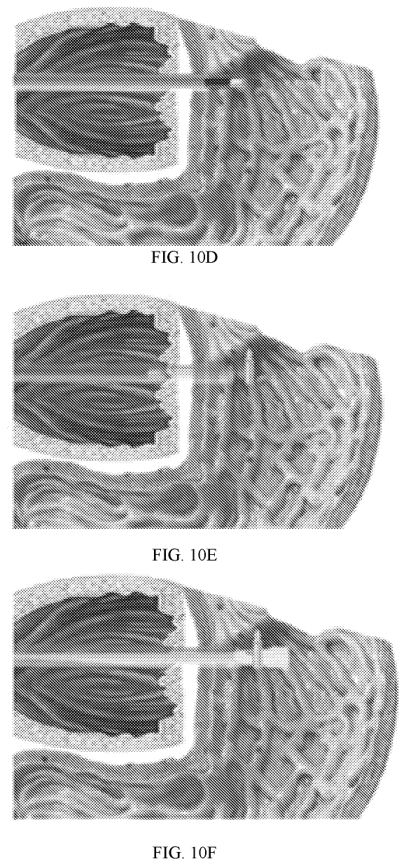

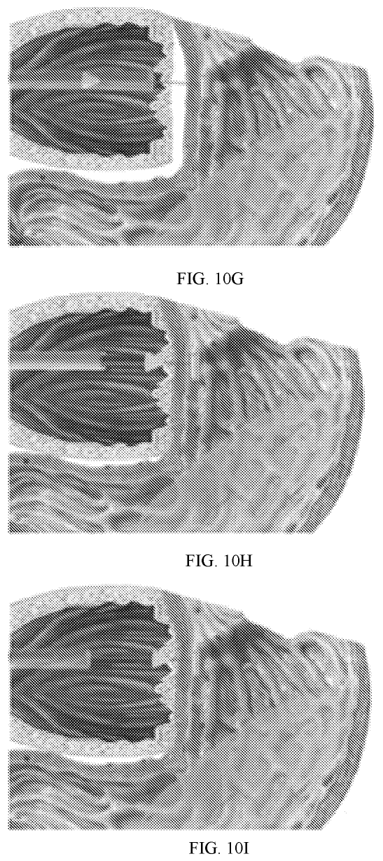

FIGS. 10A-10J depicts a process for implanting or installing an anchor via a therapeutic anchor device according to an embodiment of the present disclosure. The therapeutic EUS anchor device may be the device 100 depicts in FIG. 2.

At FIG. 10A, the EUS scope is directed down the esophagus to the stomach pouch. The needle sheath, endoscopic cannula, wire suture, and anchor are positioned within the EUS scope, such that the respective distal ends are within the stomach pouch.

At FIG. 10B, the anchor can be positioned external to the EUS scope. In some cases, the anchor can be positioned partially external to the endoscopic cannula (e.g., in cases where the one end of the anchor is tipped). In other cases, the anchor may still be completely positioned within the endoscopic cannula (e.g., in cases where the cannula end is tipped).

At FIG. 10C, the anchor and endoscopic cannula is passed into the tissue layers of the remnant stomach. In some cases, the anchor and cannula are plunged through the tissue with the assistance of EUS (e.g., the EUS probe of the EUS scope).

At FIG. 10D, the needle sheath can enter the remnant stomach or jejunum. The anchor can completely exit the endoscopic cannula. At FIG. 10E, the endoscopic cannula can retreat out of the remnant stomach, leaving the anchor and wire suture in the remnant stomach or jejunum.

At FIG. 10F, fluid can be flowed through the needle sheath and into the remnant stomach or jejunum, which can fill the remnant stomach with fluid, which can provide improved lumen visibility using the EUS. At FIG. 10G, the needle sheath can retreat from the stomach pouch. A cinch can be fed over the wire suture along with a pusher tube. The pusher tube can push the cinch down through the needle sheath and into the stomach pouch.

At FIG. 10H, the anchor can be pulled tight against the remnant stomach wall (e.g., by pulling the proximal end of the wire suture). The pusher tube can push the cinch against the stomach, stomach pouch, or jejunal walls. These pushing and pulling actions can tighten the cinch and anchor against the respective GI walls.

At FIG. 10I, the needle sheath and pusher tube can be removed from the stomach pouch. At FIG. 10J, a cinch is translated down the EUS scope. The wire suture can be guided into a cinch lumen (e.g., via a hooked wire translatable through the cinch lumen), and the cinch distal end can be positioned approximate to the cinch. The distal end of the clamp can then cut or cinch the wire suture, with excess suture retreated out through the esophagus.

### Apertured Cannula

In some cases, the endoscopic cannula can include a number of apertures defined on the distal end of the cannula. The apertures can expose the distal end of the cannula lumen to the external environment. In some cases, the fluid can be flowed through the cannula lumen, which can be used to fill the remnant stomach or jejunum with fluid. This can, for example, be used to replace the step depicted in FIG. 10F (e.g., which relies on the need sheath for flowing fluid). For example, the apertures can be sized in the range between 0.01 to 1.0 mm.

### Injection Port

In some cases, the needle sheath can include an injection port. The injection port can include a distal end, and can define an injection port lumen. The injection port can in some cases diverge away from the distal end of the needle sheath for positioning the endoscopic cannula, wire suture, anchor, and the like. The injection port lumen can flow water into the remnant stomach. This can, for example, be used to replace the step depicted in FIG. 10F. In some cases, the injection port and the distal end of the needle sheath can form a Y-connector.

### Multiple Anchors

In some cases, multiple anchors can be disposed within the endoscopic cannula simultaneously (e.g., multiple anchor-pusher assemblies lying within a single endoscope lumen or multiple endoscope lumen). This can be beneficial in cases where multiple anchors are to be implanted during a single operation. The anchors can be similarly coupled in the respective middle portions to corresponding wire sutures (e.g., such that multiple wire sutures are disposed within the endoscopic cannula). The predisposed position along the length of the cannula lumen can vary between the anchors. For example, one anchor can be disposed closer to the distal end of the cannula lumen compared to another anchor. This can allow for multiple anchors to simultaneously be located in the cannula lumen without having to increase the cannula lumen cross-sectional diameter. Further, the anchors can be implanted according to the respective positions along the cannula lumen length. For example, the anchor positioned closest to the distal end of the cannula lumen can be implanted first; the anchor second closest to the lumen distal end can be implanted second, and so forth. This can assist in mitigating any problems associated with wire suture tangling and the like.

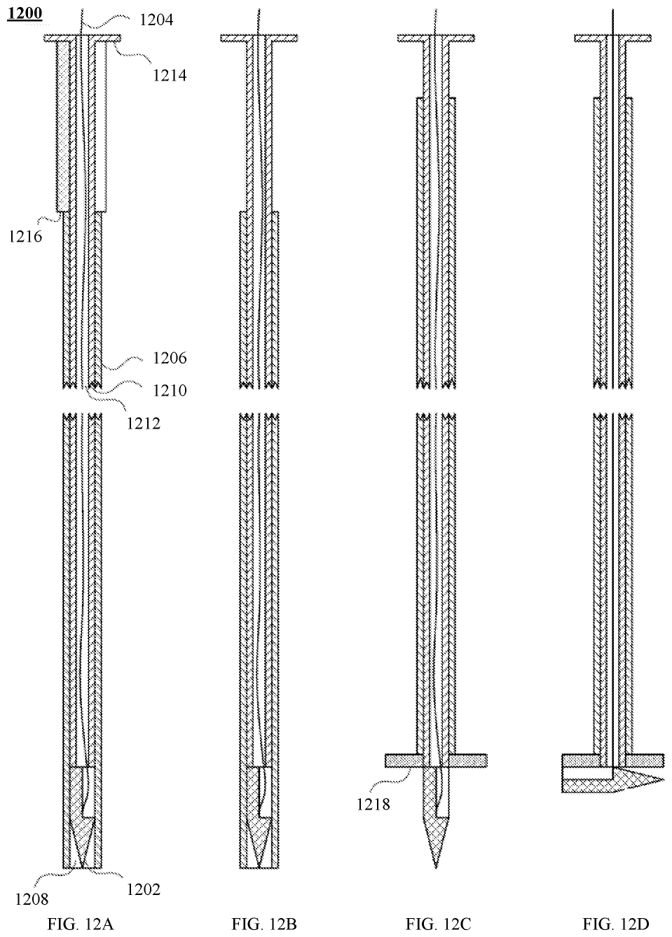

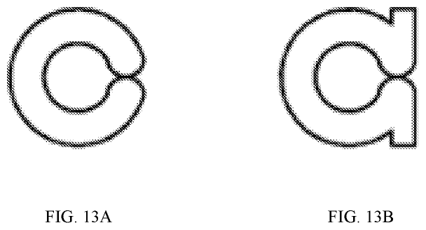

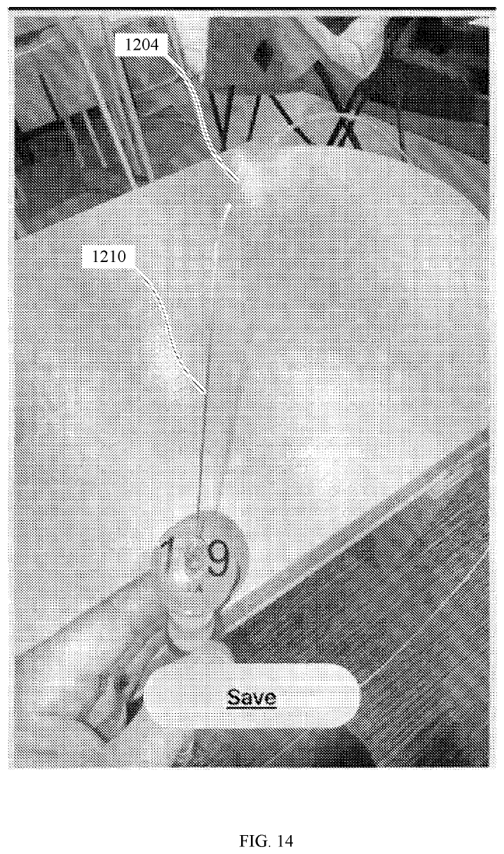

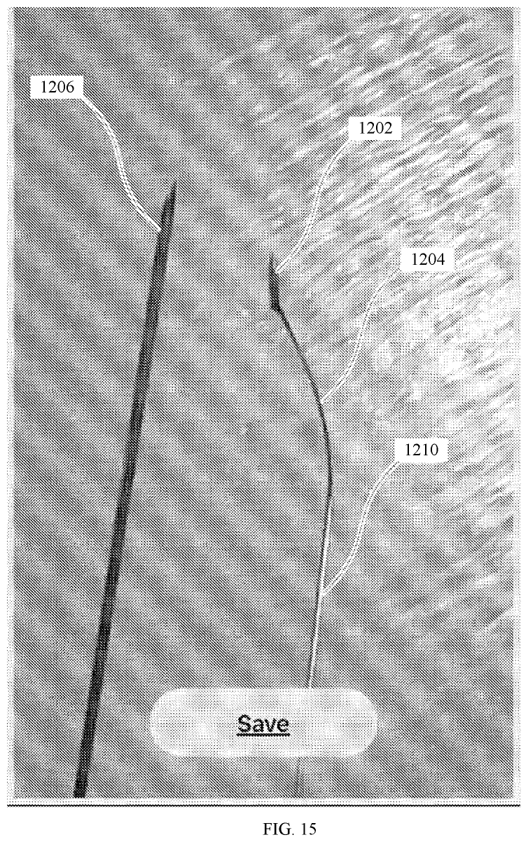

Referring now to FIGS. 12A-12D, one embodiment of the invention provides a system 1200 including an anchor 1202, a suture 1204 coupled to a medial portion of the anchor 1202; an outer cannula 1206 defining and outer-cannula central bore 1208; and an inner cannula 1210 defining an inner-cannula central bore 1212 of sufficient cross-sectional dimension to accommodate the suture 1204. Positioning the suture 1204 within the inner-cannula central bore 1212 of the inner cannula avoids friction that can occur when the suture 1204 runs between a pusher rod and outer cannula 1206.

The suture 1204 can be of a variety of materials and thicknesses as used in surgical procedures. For example, the suture 1204 can be 2.0 or 3.0 polypropylene.

The outer-cannula central bore 1208 has a sufficient cross-sectional dimension to accommodate the inner cannula 1210. The inner cannula 1210 and the outer cannula 1206 can each have a length greater than about 1 m, e.g., 1,380 mm, 1,430 mm, between about 1 m and about 1.5 m, and the like. For example, the length of cannulas 1206, 1210 can have sufficient length for use in an endoscopic procedures such as endoscopic ultrasound (EUS)-directed transgastric ERCP (EDGE), Roux-en-Y gastric bypass, gastro-jejunostomy, and the like. The length of cannulas 1206, 1210 can be the working length, e.g., the portion distal to a proximal handle that is advanceable within an endoscope.

The relative lengths of cannulas 1206, 1210 can be designed to allow the inner cannula 1210 to push the anchor 1202 beyond the outer cannula 1206, but not advance beyond the distal end of the outer cannula 1206. For example, cannulas 1206, 1210 can have a difference in length (e.g., between about 2 mm and about 30 mm, between about 2 mm and about 5 mm, etc.) reflecting the axial dimension of anchor 1202. The difference in length can vary based on the outer cannula's length.

The inner cannula 1210 can include a proximal stop shoulder 1214 at a proximal end. The proximal stop shoulder 1214 can have a cross-sectional profile larger than at least the outer-cannula central bore 1208 to prevent the proximal end of the inner cannula 1210 from entering the outer cannula 1206.

In some embodiments, the inner cannula 1210 can be a 22 G needle and the outer cannula 1206 can be a 19 G needle. However, other needle gauges can be selected, e.g., using Table 1 detailing dimensions using the Birmingham gauge. Although the cannulas 1206, 1210 are depicted in FIGS. 12A-12D as having limited clearance, the cannulas can be sized to have looser or tighter tolerances.

TABLE 1
Needle Gauges and Associated Diameters

| Gauge, | Nominal outer diameter | | Nominal inner diameter | |
|---|---|---|---|---|
| G | (inch) | (mm) | (inch) | (mm) |
| 7 | 0.180 ± 0.001 | 4.572 ± 0.025 | 0.150 ± 0.003 | 3.810 ± 0.076 |
| 8 | 0.165 ± 0.001 | 4.191 ± 0.025 | 0.135 ± 0.003 | 3.429 ± 0.076 |
| 9 | 0.148 ± 0.001 | 3.759 ± 0.025 | 0.118 ± 0.003 | 2.997 ± 0.076 |
| 10 | 0.134 ± 0.001 | 3.404 ± 0.025 | 0.106 ± 0.003 | 2.692 ± 0.076 |
| 11 | 0.120 ± 0.001 | 3.048 ± 0.025 | 0.094 ± 0.003 | 2.388 ± 0.076 |
| 12 | 0.109 ± 0.001 | 2.769 ± 0.025 | 0.085 ± 0.003 | 2.159 ± 0.076 |
| 13 | 0.095 ± 0.001 | 2.413 ± 0.025 | 0.071 ± 0.003 | 1.803 ± 0.076 |
| 14 | 0.083 ± 0.001 | 2.108 ± 0.025 | 0.063 ± 0.003 | 1.600 ± 0.076 |
| 15 | 0.0720 ± 0.0005 | 1.829 ± 0.013 | 0.0540 ± 0.0015 | 1.372 ± 0.038 |
| 16 | 0.0650 ± 0.0005 | 1.651 ± 0.013 | 0.0470 ± 0.0015 | 1.194 ± 0.038 |
| 17 | 0.0580 ± 0.0005 | 1.473 ± 0.013 | 0.0420 ± 0.0015 | 1.067 ± 0.038 |
| 18 | 0.0500 ± 0.0005 | 1.270 ± 0.013 | 0.0330 ± 0.0015 | 0.838 ± 0.038 |
| 19 | 0.0420 ± 0.0005 | 1.067 ± 0.013 | 0.0270 ± 0.0015 | 0.686 ± 0.038 |
| 20 | 0.03575 ± 0.00025 | 0.9081 ± 0.0064 | 0.02375 ± 0.00075 | 0.603 ± 0.019 |
| 21 | 0.03225 ± 0.00025 | 0.8192 ± 0.0064 | 0.02025 ± 0.00075 | 0.514 ± 0.019 |
| 22 | 0.02825 ± 0.00025 | 0.7176 ± 0.0064 | 0.01625 ± 0.00075 | 0.413 ± 0.019 |
| 22s | 0.02825 ± 0.00025 | 0.7176 ± 0.0064 | 0.00600 ± 0.00075 | 0.152 ± 0.019 |
| 23 | 0.02525 ± 0.00025 | 0.6414 ± 0.0064 | 0.01325 ± 0.00075 | 0.337 ± 0.019 |
| 24 | 0.02225 ± 0.00025 | 0.5652 ± 0.0064 | 0.01225 ± 0.00075 | 0.311 ± 0.019 |
| 25 | 0.02025 ± 0.00025 | 0.5144 ± 0.0064 | 0.01025 ± 0.00075 | 0.260 ± 0.019 |
| 26 | 0.01825 ± 0.00025 | 0.4636 ± 0.0064 | 0.01025 ± 0.00075 | 0.260 ± 0.019 |
| 26s | 0.01865 ± 0.00025 | 0.4737 ± 0.0064 | 0.00500 ± 0.00075 | 0.127 ± 0.019 |
| 27 | 0.01625 ± 0.00025 | 0.4128 ± 0.0064 | 0.00825 ± 0.00075 | 0.210 ± 0.019 |
| 28 | 0.01425 ± 0.00025 | 0.3620 ± 0.0064 | 0.00725 ± 0.00075 | 0.184 ± 0.019 |
| 29 | 0.01325 ± 0.00025 | 0.3366 ± 0.0064 | 0.00725 ± 0.00075 | 0.184 ± 0.019 |
| 30 | 0.01225 ± 0.00025 | 0.3112 ± 0.0064 | 0.00625 ± 0.00075 | 0.159 ± 0.019 |
| 31 | 0.01025 ± 0.00025 | 0.2604 ± 0.0064 | 0.00525 ± 0.00075 | 0.133 ± 0.019 |
| 32 | 0.00925 ± 0.00025 | 0.2350 ± 0.0064 | 0.00425 ± 0.00075 | 0.108 ± 0.019 |
| 33 | 0.00825 ± 0.00025 | 0.2096 ± 0.0064 | 0.00425 ± 0.00075 | 0.108 ± 0.019 |
| 34 | 0.00725 ± 0.00025 | 0.1842 ± 0.0064 | 0.00325 ± 0.00075 | 0.0826 ± 0.019 |

In some embodiments, the cannulas 1206, 1210 have polished surfaces or low-friction coatings such as polytetrafluoroethylene (PTFE) (e.g., TEFLON® available from The Chemours Company of Wilmington, Delaware). In some embodiments, a surgical lubricant (e.g., SURGILUBE® available from HR Pharmaceuticals, Inc. of York, Pennsylvania) is applied to reduce friction between the cannulas 1206, 1210.

Referring still to FIG. 12A, the system 1200 can be provided assembled as shown or can be assembled in the field. Anchor 1202 lies in the distal end of outer cannula 1206. Anchor 1202 can include a beveled distal tip to facilitate advancement through tissue to be anchored. Suture 1204 runs through the inner-cannula central bore 1212 of inner cannula 1210 to the distal end of the inner cannula 1210. A removable stopper 1216 can sit over the inner cannula 1210 to prevent distal advancement of the inner cannula 1210 and the anchor 1202 until the system 1200 is positioned appropriately.

In FIG. 12B, the removable stopper 1216 is removed.

In FIG. 12C, the inner cannula 1210 is advanced distally to push the anchor 1102 through the tissue 1118 to be anchored.

In FIG. 12D, proximal tension is applied to the suture 1204 to cause the anchor 1202 to rotate transversely and press against the tissue to be anchored. The cannulas 1206, 1210 can be removed proximally and a cinch (e.g., as described in U.S. Provisional Patent Application Ser. No. 63/275,788) can be advanced distally along the suture 1204 (e.g., using a pusher adapted and configured to rider over suture 1204).

Certain embodiments of the present disclosure can include a “deployable” anchor with a needle (e.g., a 19 G needle) placed through the linear EUS endoscopy. Such a needle can be passed from a first lumen (e.g., “lumen A”) to a second lumen (e.g., “lumen B”). Fenestration in a catheter (e.g., a 19 gauge catheter) allows contrast infusion into the second lumen (e.g., “lumen B”) for confirmation of proper position. An anchor with an attached suture can then be deployed from distal end of the catheter into the second lumen (e.g., “lumen B”). The catheter (e.g., 19 G catheter) can be removed. A cinch can be placed over a suture and passed to the first lumen (e.g., “lumen A”). The suture can be fixed and/or cut.

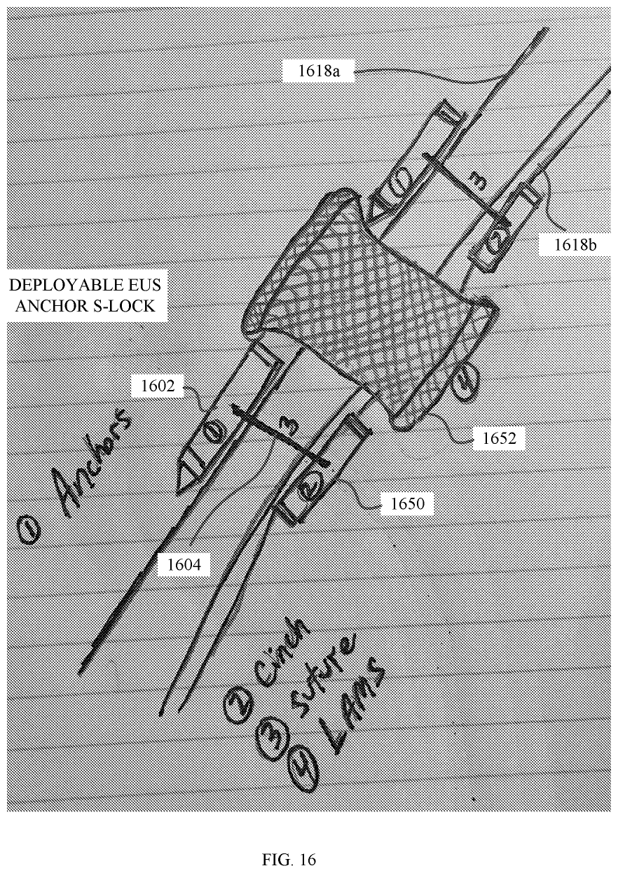

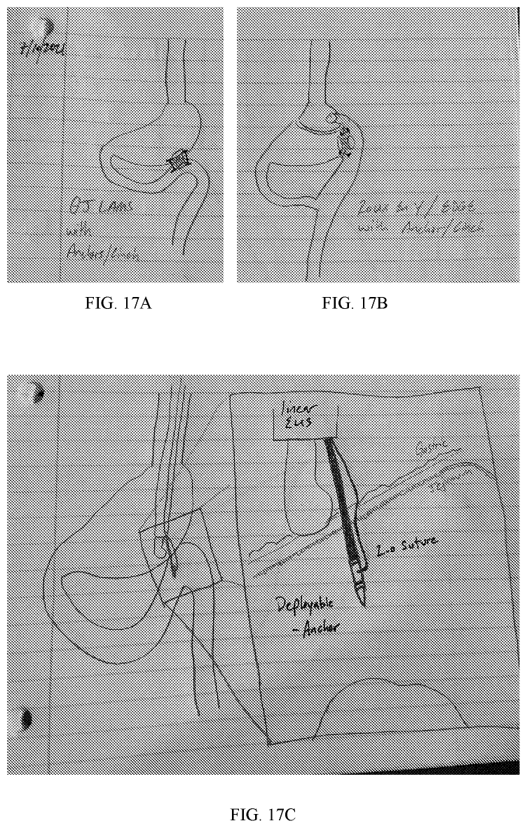

FIG. 16 illustrates a deployable “EUS Anchor S-Lock” disposed over tissue 1618a and 1618b. After using certain EUS anchoring systems and devices herein in certain applications, the end result can be depicted in FIG. 16. As illustrated, anchor 1602 is connected to a corresponding cinch 1650 via a suture 1604, thereby pressing tissue 1618a against tissue 1618b. LAMS 1652 can be further used to secure tissue 1618a to tissue 1618b.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1-20. (canceled)

21. An endoscopic ultrasound (EUS) anchoring system comprising:
an outer cannula defining an outer-cannula lumen;
an inner cannula translatable within the outer-cannula lumen and defining an inner-cannula lumen;
an anchor that is disposed at a distal end of the inner cannula and is pushable by the inner cannula out of a distal end of the outer cannula; and
a wire suture translatable through the inner-cannula lumen having a distal end coupled to the anchor.

22. The EUS anchoring system of claim 21, further comprising:
an EUS scope defining a EUS lumen; and
a cinch mechanism translatable within the EUS lumen and configured to:
cut the wire suture, forming a cut proximal end of the wire suture; and
couple the cut proximal end of the wire suture to the cinch mechanism while the distal end of the wire suture remains coupled to the anchor.

23. The EUS anchoring system of claim 22, further comprising:
a plunger included in the cinch mechanism and configured to apply a compressive force on the wire suture.

24. The EUS anchoring system of claim 22, wherein the cinch mechanism is housed within an anchor pusher handle.

25. The EUS anchoring device of claim 21, wherein the anchor comprises a first end, a second end, and a middle portion, and the distal end of the wire suture is coupled to the middle portion of the anchor.

26. The EUS anchoring system of claim 21, further comprising:
a hooked wire translatable within a clamp lumen defined by a clamp, the hooked wire having a distal end comprising a hook, wherein the hooked wire is configured to:
couple to the wire suture via the hook; and
position the wire suture into the clamp lumen by translating the hook distally away from the anchor.

27. The EUS anchoring system of claim 21, further comprising:
an EUS scope defining a EUS lumen;
wherein the outer cannula is translatable within the EUS lumen.

28. The EUS anchoring system of claim 27, wherein:
the outer cannula comprises a first outer cannula and a second outer cannula;
the second outer cannula is translatable within the EUS lumen;
the inner cannula is arranged and disposed to pass from the first outer cannula to the second outer cannula, and is translatable within the second outer cannula; and
the anchor is arranged and disposed to hinge around the coupling with the wire suture once exiting the second outer cannula.

29. The EUS anchoring system of claim 21, wherein the outer cannula further defines a fluid injection port divergent from a distal portion of the outer cannula.

30. The EUS anchoring system of claim 29, wherein the fluid injection port and the distal portion form a Y connect.

31. The EUS anchoring system of claim 21, wherein the outer cannula further defines a plurality of apertures along a distal portion of the outer cannula.

32. The EUS anchoring system of claim 21, further comprising:
a second anchor translatable within the outer lumen and egressable from the distal end of the outer lumen.

33. The EUS anchoring system of claim 32, further comprising a second wire suture translatable through the inner-cannula lumen having a distal end coupled to the second anchor.

34. The EUS anchoring system of claim 32, wherein the anchor and the second anchor are predisposed at different positions along a length of the inner-cannula lumen.

35. The EUS anchoring system of claim 21, wherein a length of the anchor is parallel to a length of the inner-cannula lumen when disposed within the outer cannula.

36. An anchor delivery system comprising:
an anchor;
a wire suture coupled to the anchor;
an outer cannula defining an outer-cannula central bore; and
an inner cannula defining an inner-cannula central bore,
wherein the inner-cannula central bore has a cross-sectional dimension configured to accommodate the suture; the outer-cannula central bore has a cross-sectional dimension configured to accommodate the inner cannula; the inner cannula has a length greater than about 1 m; and
wherein the inner cannula further comprises a proximal stop shoulder at a proximal end, the proximal stop shoulder having a cross-sectional profile larger than a diameter of the outer-cannula central bore to prevent the proximal end from entering the outer cannula.

37. The anchor delivery system of claim 36, further comprising a removable stopper adapted and configured to be removably mounted on an outer surface of the inner cannula to prevent advancement of the inner cannula and the anchor distal to the inner cannula from advancing within the outer cannula.

38. A method for implanting an anchor using the EUS anchoring system of claim 21, comprising the steps of:
(a) directing an EUS scope towards a first section of tissue, the EUS scope including an EUS lumen extending therethrough;
(b) positioning the outer cannula, the inner cannula, the wire suture, and the anchor within the EUS lumen;
(c) extending the inner cannula, the wire suture, and the anchor from the EUS scope, through the first section of tissue, and into a second section of tissue;
(d) hinging the anchor around the coupling with the wire suture; and
(e) retreating the inner cannula from the tissue layers, thereby leaving the anchor and the wire suture in the second section of tissue.

39. The method of claim 38, further comprising the steps of:
extending the outer cannula from the EUS scope, through the first section of tissue, and into the second section of tissue;
flowing a fluid through the outer cannula and into the second section of tissue; and then
retreating the outer cannula from the second section of tissue.

40. The method of claim 38, further comprising the steps of:
(f) feeding a cinch over the wire suture along with a pusher tube;
(g) pulling the anchor tight against the tissue layers by pulling a proximal end of the wire suture;
(h) pushing the cinch against the tissue layers by the pusher tube;
(i) cutting the wire suture via the cinch mechanism; and
(j) removing the pusher tube.
